(19) United States
(12) Patent Application Publication
(10) Pub. No.: US 2026/0212495 A1
(43) Pub. Date: Jul. 23, 2026

(54) AUTOMATIZED DETECTION OF INTESTINAL INFLAMMATION IN CROHN'S DISEASE USING CONVOLUTIONAL NEURAL NETWORK

(71) Applicants: Sheba Impact Ltd., Ramat-Gan (IL); Afeka Yissumim Ltd., Tel-Aviv (IL)

(72) Inventors: Dan CARTER, Ganei Tikva (IL); Oshrit HOFFER, Kiryat-Ono (IL)

(73) Assignees: Sheba Impact Ltd., Ramat-Gan (IL); Afeka Yissumim Ltd., Tel-Aviv (IL)

(21) Appl. No.: 19/139,414

(22) PCT Filed: Dec. 14, 2023
(86) PCT No.: PCT/IL2023/051277

§ 371 Date: Jun. 16, 2025

Related U.S. Application Data
(60) Provisional application No. 63/432,721, filed on Dec. 15, 2022.

(51) Int. Cl. G06T 7/00; A61B 8/00; A61B 8/08; G06N 3/08; G06T 7/11; G06V 10/764; G06V 10/774; G06V 10/82

(52) U.S. Cl. CPC G06T 7/0012; A61B 8/08; A61B 8/461; G06N 3/08; G06T 7/11; G06V 10/764; G06V 10/82; G06T 2207/10132; G06T 2207/20081; G06T 2207/20084; G06T 2207/30028; G06V 10/774; G06V 2201/03

(57) ABSTRACT

The invention relates to system and methods for predicting and/or diagnosing of IBD from ultrasound images according to one or more of diagnostic signs.

### RELATED APPLICATION/S

This application claims the benefit of priority of U.S. Provisional Patent Application No. 63/432,721 filed on 15 Dec. 2022, the contents of which are incorporated herein by reference in their entirety.

### FIELD AND BACKGROUND OF THE INVENTION

The present invention, in some embodiments thereof, relates to an automatized system and method for the detection of intestinal inflammation in intestinal diseases (as inflammatory disorders like Crohn's Disease (CD), Ulcerative colitis), celiac disease, eosinophilic disorders of the intestinal tract, intestinal infections and cancer) using convolutional neural network.

The use of Intestinal ultrasound (IUS) for the initial evaluation, diagnosis and follow-up of intestinal disorders, like inflammatory bowel disease, IBD, is steadily growing. It has been shown that thickening of the bowel wall is the most significant ultrasonographic feature of IBD, found to be a valid marker of inflammation in IBD. While access to educational platforms of IUS is feasible, novice US operators lack experience in performing and interpreting IUS.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. A system, comprising:
a. an ultrasound sensing device; and
b. a computing device, the computing device comprising:
i. a communication interface configured to directly receive ultrasound echo data of at least a portion of an intestine sensed by the ultrasound sensing device from a person, the received ultrasound echo data of said at least a portion of an intestine comprising a sequence of ultrasound images of said at least a portion of an intestine;
ii. a memory configured for:
A. storing a representation of a diagnostic protocol for IBD predicting a diagnosis of IBD for a patient based on the presence of a plurality of diagnostic signs in ultrasound images captured from a patient, and
B. storing one or more neural networks trained to identify diagnostic signs among the plurality of diagnostic signs in ultrasound images of at least a portion of an intestine;
iii. a processor configured to:
C. applying to each received ultrasound image of said at least a portion of an intestine at least one of the one or more trained neural networks to identify as present or absent in the received ultrasound image diagnostic signs of IBD among the plurality of diagnostic signs of IBD, until the plurality of signs are all identified as present or absent in the person, causing the capture of additional ultrasound images from the person by the ultrasound sensing device, and when all of the plurality of signs are identified as present or absent in the person, using the signs identified as present or absent to evaluate the represented diagnostic protocol for IBD to obtain a tentative diagnosis of IBD of the person; and
iv. a display device configured to causing the tentative diagnosis of IBD of the person to be displayed.

Example 2. The system according to example 1, wherein said plurality of diagnostic signs comprise one or more of bowel wall thickening greater than 3 mm, increased bowel wall blood flow as detected by doppler, mesenteric fat proliferation, disappearance of bowel wall layers, enlarged lymph nodes in the mesenteric fat and appearance of intraluminal complications and/or extramural complications.

Example 3. The system according to example 1 or example 2, wherein the ultrasound sensing device comprises a transducer.

Example 4. The system according to any one of examples 1-3, wherein one of the plurality of diagnostic signs is a compound sign that depends on two or more other of the plurality of diagnostic signs, wherein the processor is further configured for determining whether the compound sign is present or absent based on whether the diagnostic signs on which the compound sign depends are identified as present or absent.

Example 5. The system according to any one of examples 1-4, wherein at least one of the trained neural networks stored by the memory comprises a Mobile U-Net.

Example 6. One or more instances of computer-readable media collectively having contents configured to cause a computing system to perform a method, the method comprising:
a. accessing a set of diagnostic signs of IBD involved in a diagnostic protocol for IBD;
b. until the presence or absence of each of the diagnostic signs of IBD of the set has been identified in ultrasound images of a patient:
i. causing an ultrasound image of at least a part of an intestine to be captured from the patient;
ii. applying to the captured ultrasound image of at least a part of an intestine a trained machine learning model among one or more trained machine learning models to identify the presence or absence of one or more diagnostic signs of IBD of the set;
iii. evaluating the diagnostic protocol for IBD with respect to the identified presence or absence of each of the set of diagnostic signs of IBD to obtain a preliminary diagnosis for IBD; and
iii. storing the preliminary diagnosis.

Example 7. The one or more instances of computer-readable media according to example 6, wherein said set of diagnostic signs comprise one or more of bowel wall thickening greater than 3 mm, increased bowel wall blood flow as detected by doppler, mesenteric fat proliferation, disappearance of bowel wall layers, enlarged lymph nodes in the mesenteric fat and appearance of intraluminal complications and/or extramural complications.

Example 8. The one or more instances of computer-readable media according to example 6 or example 7, the method further comprising training at least one machine learning model to identify the presence or absence of one or more diagnostic signs of the set.

Example 9. The one or more instances of computer-readable media according to any one of examples 6-8, the method further comprising causing the obtained preliminary diagnosis to be displayed.

Example 10. The one or more instances of computer-readable media according to any one of examples 6-9, wherein one of the set of diagnostic signs is a compound sign that depends on two or more other of the set of diagnostic signs, the method further comprising determining whether the compound sign is present or absent based on whether the diagnostic signs on which the compound sign depends are identified as present or absent.

Example 11. A method in a computing system, the method comprising:
a. accessing a set of diagnostic signs of IBD involved in a diagnostic protocol for IBD;
b. until the presence or absence of each of the diagnostic signs of the set has been identified in ultrasound images of a patient:
i. causing an ultrasound image to be captured from the patient;
ii. applying to the captured ultrasound image a convolutional neural network among one or more trained convolutional neural networks to identify the presence or absence of one or more diagnostic signs of the set;
iii. evaluating the diagnostic protocol with respect to the identified presence or absence of each of the set of diagnostic signs to obtain a preliminary diagnosis; and
iv. storing the preliminary diagnosis.

Example 12. The one or more instances of computer-readable media according to example 11, wherein said set of diagnostic signs comprise one or more of bowel wall thickening greater than 3 mm, increased bowel wall blood flow as detected by doppler, mesenteric fat proliferation, disappearance of bowel wall layers, enlarged lymph nodes in the mesenteric fat and appearance of intraluminal complications and/or extramural complications.

Example 13. The method according to example 11 or example 12, further comprising training at least one convolutional neural network to identify the presence or absence of one or more diagnostic signs of the set.

Example 14. The method according to any one of examples 11-13, further comprising causing the obtained preliminary diagnosis to be displayed.

Example 15. The method according to any one of examples 11-14, wherein one of the set of diagnostic signs is a compound sign that depends on two or more other of the set of diagnostic signs, the method further comprising: determining whether the compound sign is present or absent based on whether the diagnostic signs on which the compound sign depends are identified as present or absent.

Example 16. The system according to any one of examples 11-15, wherein at least one of the one or more convolutional neural networks comprises a Mobile U-Net.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as MATLAB, Python, Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of an exemplary sequence of an exemplary study, according to some embodiments of the invention;

FIG. 2 is a representation of an exemplary cropped image, according to some embodiments of the invention;

FIG. 3A-D are representations of exemplary manual cropped images, according to some embodiments of the invention;

FIG. 3E are representation of an original image and a segmented image, according to some embodiments of the invention.

FIG. 4 is an exemplary confusion matrix, according to some embodiments of the invention;

FIG. 5 is an exemplary bowel wall thickness classification ROC curve, according to some embodiments of the invention;

FIG. 6 is a flowchart of an exemplary decision of treatment method, according to some embodiments of the invention;

FIG. 7 is a flowchart of an exemplary home monitoring method, according to some embodiments of the invention; and

FIG. 8 is a flowchart of an exemplary method of the actions performed in an exemplary IBD unit meeting following the receipt of the results of the examinations, according to some embodiments of the invention.

### DESCRIPTION OF SPECIFIC EMBODIMENTS OF THE INVENTION

The present invention, in some embodiments thereof, relates to an automatized system and method for the detection of intestinal inflammation in intestinal diseases (as inflammatory disorders like Crohn's Disease (CD), Ulcerative colitis), celiac disease, eosinophilic disorders of the intestinal tract, intestinal infections and cancer) using convolutional neural network.

### Overview

An aspect of some embodiments of the invention relates to recognition of ultra-sonographic signs of intestinal inflammation in intestinal diseases (for example inflammatory disorders like Crohn's Disease (CD) and Ulcerative colitis; other intestinal diseases like celiac disease, eosinophilic disorders of the intestinal tract, intestinal infections and cancer) IBD and its activity by deep learning using a convolutional neural network. In some embodiments, the recognition is provided with high levels of accuracy when compared to known methods, for example, the recognition is provided with a sensitivity of about 90% and a specificity of about 95.6% when compared to CT and/or MRI [REF. 17].

In some embodiments, a potential advantage of the system and method is that an artificial intelligence (AI)-based operator supporting system that automatically detects bowel wall inflammation may potentially simplify the use of Intestinal ultrasound (IUS) by less experienced operators. In some embodiments, the system comprises an AI module configured to distinguish bowel wall thickening, which is a surrogate of bowel inflammation, from normal bowel wall thickness in images of IUS. In some embodiments, the system is configured to perform automatic segmentation processes and automatic measurements on the US image. In some embodiments, segmentation and measurements are used in the process of detection of bowel wall inflammation. In some embodiments, a potential advantage of utilizing automated segmentation processes and automated measuring is that it can potentially further increase the levels of accuracy when compared to known methods.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### INTRODUCTION

Technological advances in the field of ultrasonography, as well as growing experience of ultrsonographers, have contributed to the advent of ultrasound as a clinically important, non-invasive imaging modality in inflammatory bowel disease (IBD) [Ref 1]. High-resolution assessment of the bowel layers, as well as visualization of possible intestinal and extraintestinal pathologies (such as stenosis, abscesses or fistulas) can be attained plainly using intestinal US (IUS) [Ref 2]. IUS can be used for the initial evaluation and follow-up of patients with clinically suspected IBD [Refs 3 and 4]. Thickening of the bowel wall is the most significant ultrasonographic feature of CD, found to be a valid marker of inflammation in CD [Ref 5]. Transmural remission of the small and large bowel is defined by bowel wall thickness≤3 mm [Ref 6]. Other ultrasonographic signs of active disease include pseudo stratification of the bowel wall, mesenteric and adipose hypertrophy, enlargement of lymph nodes and enhancement of blood flow in the bowel wall [Ref 7]. Transmural healing [TH] has become a potential therapeutic target in IBD, as it probably reflects a pronounced and comprehensive endpoint for treatment and healing [Ref 8]. IUS can be a useful tool in such treat-to-target strategy, as an objective monitoring tool for tight control of disease activity. Results from recent studies have indicated that IUS is a promising research tool for assessment of disease control, and that IUS results can potentially be used as primary endpoints in large studies assessing drug efficacy [Refs 9, 10, 11]. The performance of IUS requires expertise, and although its use is increasing, the accessibility of IUS is still limited. Moreover, the number of novice IUS performers with limited experience is continuously growing. Therefore, an artificial intelligence-based operator supporting system that automatically detects bowel wall inflammation will potentially facilitate the use of IUS by less experienced operators. This same system can theoretically be used also as an objective tool for assessment of transmural response or transmural healing in medical studies using central reading platforms. Advances in the field of computerized image processing support the development of deep learning models that use multilayer neural networks. Supervised learning techniques, which learn a mapping from input data to output from a set of training examples, have shown great promise in medical image analysis. With improvements in computer hardware, it has become feasible to train more and more complex models on more data. In recent years, the use of supervised learning in image segmentation, recognition, and registration has accelerated. The deep learning algorithms learn on their own which features are best for the computational task [Ref 12]. Convolutional Neural Network (CNN) is an artificial neural network that uses images as an input. The use of CNN is rapidly expanding in medicine. In the field of endoscopic ultrasound, it has been used to identify and distinguish benign and malignant liver masses, pancreatic cystic lesions and for the diagnosis of autoimmune pancreatitis [Refs 13, 14, 15]. Therefore, an aspect of some embodiments of the invention relates to using an image dataset to develop and validate a deep learning module, based on a pre-trained CNN that can distinguish bowel wall thickening (a surrogate of bowel inflammation) from normal bowel images of IUS.

### Exemplary Methods

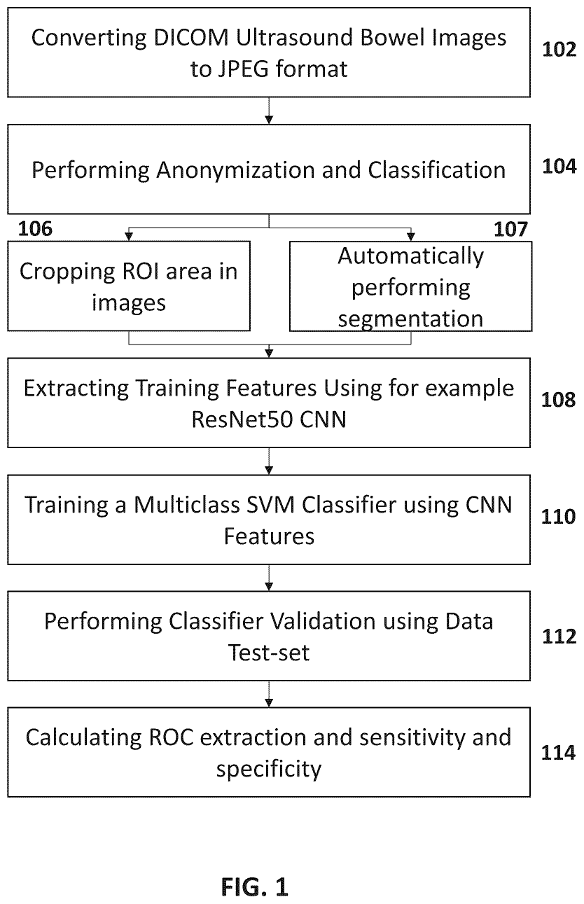

Referring now to FIG. 1, showing a flowchart of an exemplary sequence of an exemplary study, according to some embodiments of the invention.

In some embodiments, image datasets are collected to develop and validate a convolutional neural network module for distinguishing bowel wall thickening >3 mm, which is a surrogate of bowel inflammation, from normal bowel thickness in images of IUS. In some embodiments, other ultrasonographic signs of inflammation include increased bowel wall blood flow as detected by doppler, mesenteric fat proliferation, disappearance of bowel wall layers (stratification), enlarged lymph nodes in the mesenteric fat, and appearance of intraluminal complications (structures with or without presetnotic dilations) and/or extramural complications (fistulas, abscesses).

In some embodiments, in general, an exemplary sequence of an exemplary study comprises: Converting Digital Imaging and Communications (DICOM) Ultrasound Bowel Images to JPEG format 102; Performing anonymization and classification of the data 104; Cropping ROI area in images 106 or Performing segmentation of the images 107; Extracting Training Features Using ResNet50 CNN 108; Training a Multiclass SVM Classifier using CNN Features 110; Performing Classifier Validation using Data Test-set 112; and Calculating ROC extraction and sensitivity and specificity 114. Further information regarding each the above will be provided below.

### Exemplary Source of Images

Consecutive images from 308 IBD patients, who underwent IUS as part of their routine assessment and follow-up were collected. Images were converted to JPEG format 102.

### Exemplary Intestinal Ultrasound

Examinations were performed by two experienced IUS experts (D.C., A.A.; >1000 exams performed) using a GE S8 ultrasound machine (USA). The exam started with a low frequency (1-6 MHz or 2-9 MHz) curved array transducer enabling all abdominal quadrants to be examined, followed by examination using a high-resolution linear array transducer (6-15 MHz) for detailed examination of the bowel wall structure. All examinations were performed without any preceding preparation, using a consistent technique and protocol, beginning the examination with terminal ileum and the proximal to distal colon, followed by complete examination of the small bowel. Assessment was performed for features of inflammation; especially bowel walls thickness (BWT) (>3 mm). BWT≤3 mm has a significant association with endoscopic remission, transmural remission as measured by MRE and normal C-reactive protein levels.

### Preprocessing

The collected images were taken using both low- and high-resolution transducers in longitudinal and cross-sectional planes. Only images of the terminal ileum and colon (without the rectum) were used.

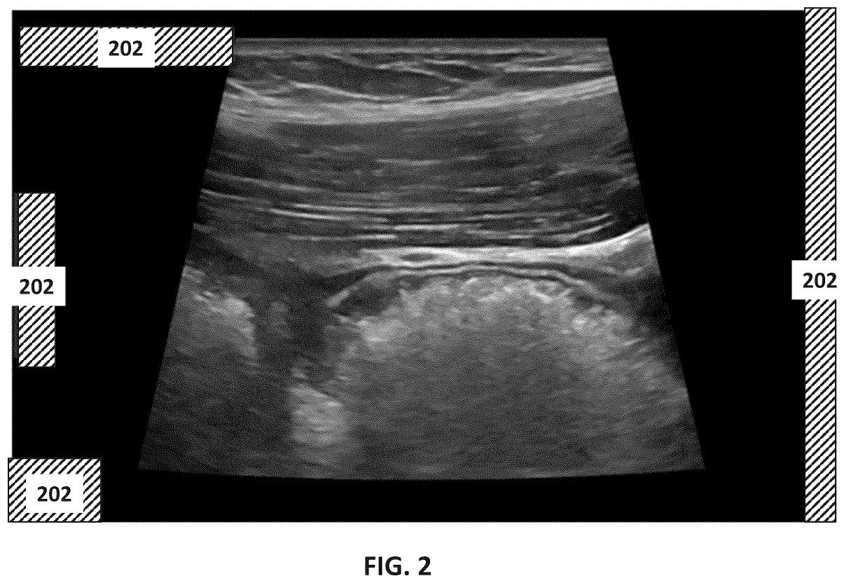

All Images were anonymized by cropping the metadata areas 104, as shown for example in FIG. 2, where cropped areas 202 are schematically shown within an US image. Subsequently, images were reviewed by D.C. and A.A and categorized into 2 groups based on the positive or negative visualization of bowel wall thickening. In some embodiments, the anonymization process is automatic. In some embodiments, the system comprises dedicated software comprising instructions to perform an identification of the metadata areas and performing automatic anonymization.

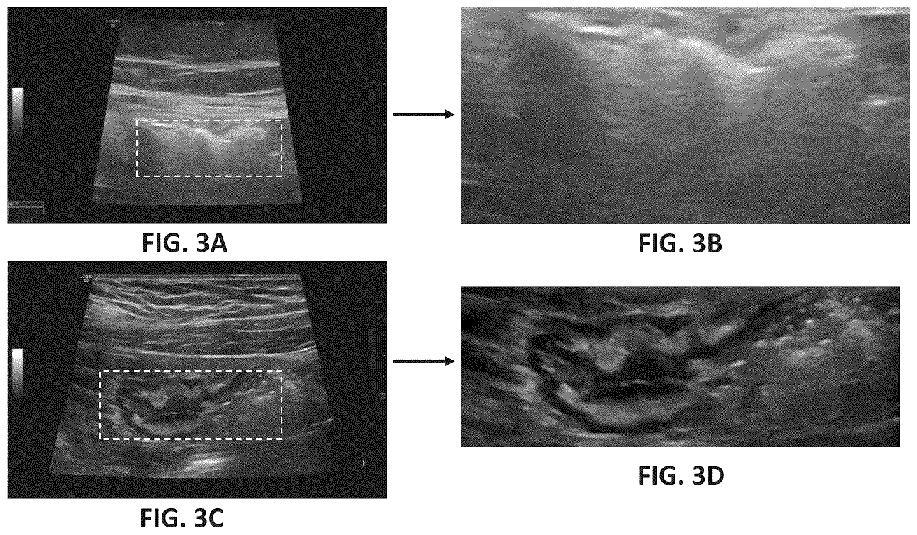

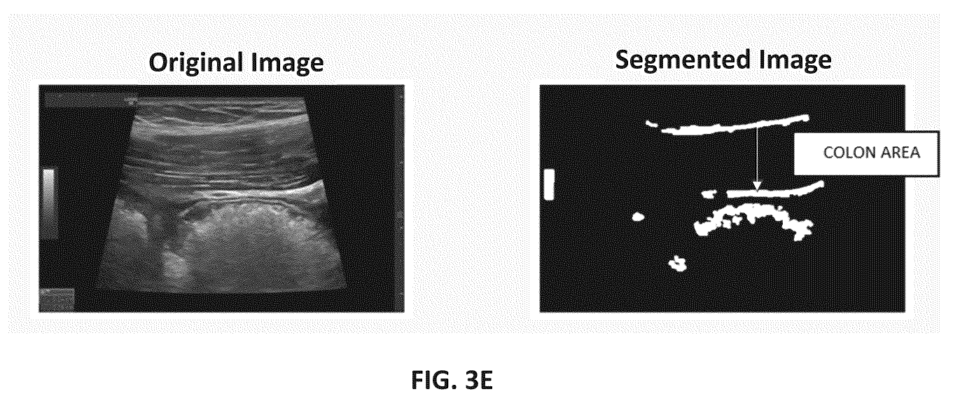

Manual cropping 106 was performed for removing the unnecessary image portions (abdominal wall), which usually includes the upper 20-30% of the image, as schematically shown for example in FIGS. 3a-d. FIGS. 3a and 3c show original images while FIGS. 3b and 3d show examples of cropped ROI areas, respectively.

In some embodiments, additionally or alternatively to performing a manual cropping 106, the system is configured for performing an automatic segmentation of the images.

In some embodiments, the segmentation comprises one or more (meaning one or a combination of) of the following actions:

Exemplary Segmentation Methods of the Colon from Ultrasound Images

### Image Preprocessing:

Filtering Techniques: In some embodiments, the methods include applying filters such as Gaussian, median, or morphological filters to enhance the image quality and reduce noise of the one or more images.

Contrast Enhancement: In some embodiments, the methods include improving the visibility of colon structures by adjusting the image contrast through techniques like histogram equalization. In some embodiments, filtering and contrast enhancements are both performed. In some embodiments, only one of the two actions is performed.

### Edge Detection:

Canny Edge Detector: In some embodiments, the method comprises identifying edges in the ultrasound images, which can help in delineating colon boundaries.

Sobel Operator: In some embodiments, the method comprises computing gradient magnitudes to highlight edges, aiding in the extraction of relevant features.

In some embodiments, performing Canny Edge Detection and performing Sobel Operator are both performed. In some embodiments, only one of the two actions is performed.

### Feature Extraction:

Texture Analysis: In some embodiments, the method comprises extracting textural features, like co-occurrence matrices or Gabor features, to characterize colon tissue patterns.

Shape Descriptors: In some embodiments, the method comprises utilizing shape-based features, such as circularity or elongation, to capture the geometry of potential colonic structures.

In some embodiments, texture analysis and using shape descriptors are both performed and/or used. In some embodiments, only one of the two actions is performed/used.

### Machine Learning Approaches:

Supervised Learning: In some embodiments, the segmentation methods utilize machine learning, and more specifically, supervised machine learning. In some embodiments, the method comprises training a classifier (e.g., Support Vector Machines or Random Forests) using labeled datasets with annotated colon regions to predict colon presence in new images.

Deep Learning: In some embodiments, Convolutional Neural Networks (CNNs) are used since they can automatically learn hierarchical features from ultrasound images, providing high accuracy in colon detection.

In some embodiments, Supervised Learning and Deep Learning are both used. In some embodiments, only one of the two methods is used.

### Region of Interest (ROI) Selection:

Segmentation Techniques: In some embodiments, as part of the automated segmentation process, segmentation algorithms like region-growing or watershed segmentation are used to isolate the colon region and discard irrelevant structures.

Active Contour Models: In some embodiments, active contour models are used, which employ snakes or level sets to iteratively refine the boundaries of the colon region.

### Registration Techniques:

Image Registration: In some embodiments, the method comprises aligning ultrasound images with a reference or standard colon model, facilitating accurate comparison and detection.

Temporal Registration: In some embodiments, the method comprises accounting for motion artifacts in dynamic ultrasound sequences, ensuring consistent detection across frames.

### Ensemble Methods:

Combination of Classifiers: In some embodiments, the method comprises combining the predictions of multiple classifiers or models to enhance overall accuracy and robustness.

### Post-Processing:

In some embodiments, independently of the action of segmentation performed, for example as explained above, a post-processing method is performed, as will be further explained below.

Morphological Operations: In some embodiments, the post-processing method comprises applying operations like dilation or erosion to refine the detected colon region and improve segmentation accuracy.

Connected Component Analysis: In some embodiments, the post-processing method comprises identifying and analyzing connected regions, helping to remove isolated noise and improve the accuracy of colon detection.

It should be noted that the effectiveness of these methods may vary based on the specific characteristics of the ultrasound images, the quality of the data, and the complexity of the colonic structures being targeted. In some embodiments, experimentation and validation on diverse datasets are performed for determining the most suitable approach for a given application.

In some embodiments, the aforementioned method is configured for detecting colons in ultrasound images through a series of image-processing steps. In some embodiments, it starts by reading an ultrasound image, converting it to grayscale if necessary, and optionally applying preprocessing techniques for image enhancement. In some embodiments, the core segmentation process involves applying thresholds to the image using, for example, Otsu's method to create a binary representation. In some embodiments, to reduce noise, morphological operations, specifically closing with a disk-shaped structuring element, are employed. In some embodiments, connected component analysis labels distinct regions, and small components, potentially noise, are filtered out based on a defined area threshold. In some embodiments, the resulting binary image represents the segmented colon, as schematically shown in FIG. 3e.

In some embodiments, the effectiveness of the method is influenced by the quality and characteristics of the input images. In some embodiments, the code provides a foundation for further refinement, encouraging users to tailor parameters and additional processing steps to their specific dataset. In some embodiments, a potential advantage of the method is that it potentially serves as a starting point for colon detection in ultrasound images, offering flexibility for adaptation and optimization based on the nuances of the imaging data at hand.

### Image Processing

For the training phase, 80% of the images were used and fed them into a residual convolutional neural network (RestNet50). ResNet50 was used for feature extraction 108. Without being bound to theory, ResNet50 takes inputs of size 224×224; thus, each image was resized to match this restriction. Features are extracted from each image using the pretrained network and a support vector machine (SVM) model is constructed based on those feature vectors and their associated labels [Ref 16]. In some embodiments, texture parameters used for the pretraining were one or more of entropy, local entropy, skewness, Kurtosis and concurrence matrix.

For the classification phase, the remaining 20% of the cropped images were used and features were extracted using the pretrained network. Then, the label of each image is predicted using the SVM model trained against the labeled images 110.

### Statistics

The main performance measures were accuracy, sensitivity, and specificity 112. The receiver operating characteristic (ROC) and area under the ROC curve (AUC) were calculated to assess the degree of discrimination 114.

### Exemplary Results

The clinical and demographic data of the study population are presented in table 1, below.

| | |
|---|---|
| Patients, n | 308 |

Gender

| | | |
|---|---|---|
| Male, n (%) | 155 | (50.3%) |
| Female, n (%) | 153 | (49.7%) |
| Age, Mean (−+ SD) | 39.5 | (15.8) |
| Disease Duration, Median (IQR) | 6 | (2-13) |

Disease extent - n (%)

| | | |
|---|---|---|
| L1- Small Bowel | 202 | (65.5) |
| L2- Colon | 21 | (7) |
| L3- Ileocolon | 85 | (27.5) |

Disease behavior - n (%)

| | | |
|---|---|---|
| B1- non structuring non penetrating | 170 | (55) |
| B2-Structuring | 70 | (23) |
| B3-Penetrating | 68 | (22) |
| Perianal Disease | 53 | (17.2) |
| Prior disease related abdominal surgery (%) | 78 | (25.3) |

Smoking, n (%)

| | | |
|---|---|---|
| Never | 206 | (67) |
| Active smoker | 53 | (17.2) |
| Past smoker | 35 | (11.3) |
| Unknown | 14 | (4.5) |
| Receiving biologic therapy, n (%) | 210 | (68.18) |

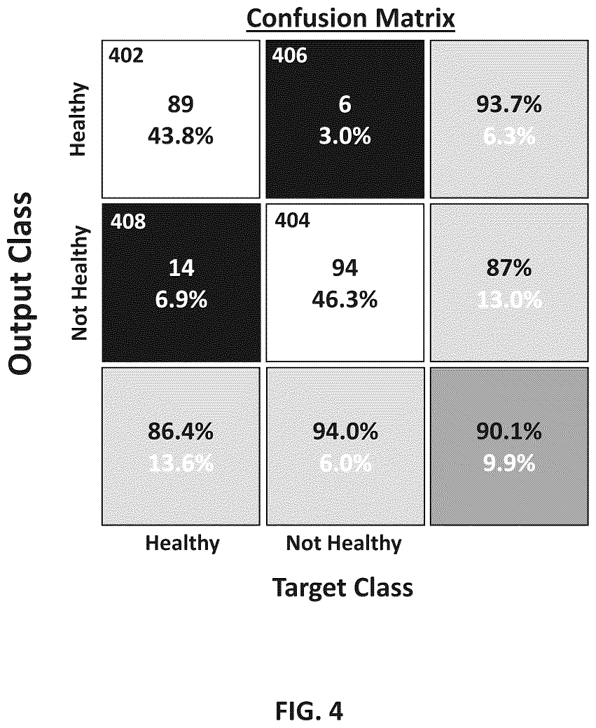

The data set consisted of 1008 images, distributed uniformly (50% normal images 50% abnormal images). Execution of the training phase and the classification phase was performed using 805 and 203 images, respectively. The confusion matrix results are presented in FIG. 4. The rows denote the true image values, and the columns denote predicted image values of the classification of thickening, which are 90.1%, 86.4%, and 94%, respectively. Out of 108 non-healthy predictions, 87% were correct and 13% were misclassified. Out of 103 healthy cases, 86.4% (sensitivity value) were correctly classified as healthy and 13.6% were diagnosed as non-healthy. Out of 100 non-healthy cases, 94% (specificity value) were correctly classified as non-healthy and 6% were classified as healthy. Overall, 90.1% (accuracy value) of the CNN diagnosis were correct and 9.9% were wrong.

In FIG. 4, the first two diagonal cells 402/404 express the number and percentage of correct classifications by the trained network, denoted by white squares. 89 bowel images are correctly classified as healthy (402). This corresponds to 43.8% of all 203 bowel images. Similarly, 94 cases are correctly classified as non-healthy (404). This corresponds to 46.3% of all bowel images. Six of the non-healthy bowel images were incorrectly classified as healthy, denoted by a black box 406, and this corresponds to 3% of all 203 bowel images in the data. Similarly, 14 of the healthy bowel images were incorrectly classified as non-healthy, denoted by a black box 408, and this corresponds to 6.9% of all data.

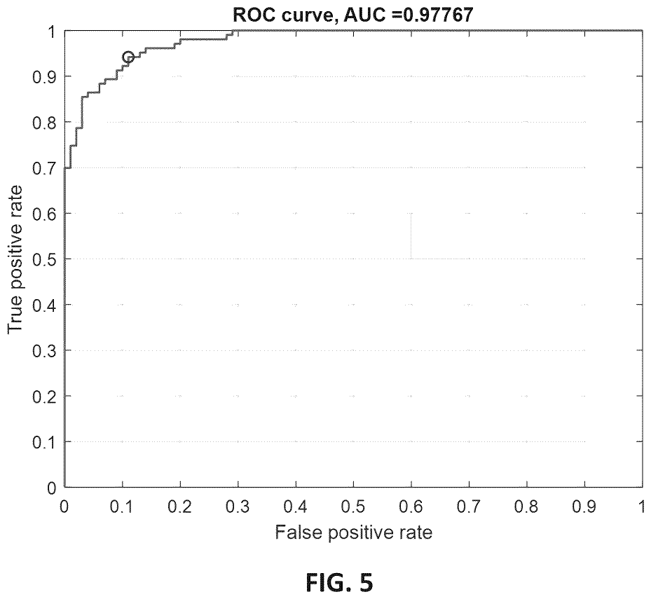

The network exhibited an area under the curve (AUC) of 0.9777 in this task. The bowel wall thickness classification ROC curve is presented, for example, in FIG. 5.

### Discussion of the Results

The above-mentioned results demonstrated that deep learning using a convolutional neural network attained accuracy in recognition of ultra-sonographic signs of IBD and its activity.

IUS is a major modality for diagnosis and monitoring of IBD, which can be performed as a point of care exam, without any special preparation. The accuracy of IUS for the detection of inflammation is similar to that of magnetic resonance imaging and computed tomography [Ref 17], with an important advantage of lack of radiation, low costs and high patient satisfaction [Ref 18]. When performed without prior preparation, it is most accurate for the detection of inflammatory changes in the terminal ileum and colon [Ref 19]. High inter and intra observer agreement rates for detection of inflammation where demonstrated when the exam was performed by IUS experts [Refs 20, 21]. However, the accuracy of IUS is operator dependent, and may relate of the operator's experience. Although IUS has been widely accepted as a bedside point of care imaging modality that is performed by gastroenterologist experts, IUS is usually not incorporated to the training curriculum of gastroenterology. Though IUS training has become feasible, most new IUS operators have limited experience in the performance and interpretation of IUS.

In some embodiments, a potential advantage of the AI system, which is optionally an automated AI system, is that it successfully augmented IUS image interpretation, and may potentially increase the accuracy of IUS interpretation and results in higher acceptability due to higher self-confidence of less experienced operators.

In some embodiments, another potential advantage is the potential use of the automatized detection of bowel inflammation related to the impending use of IUS as a significant outcome measure in IBD research.

In some embodiments, the use of AI can potentially standardize the IUS imaging interpretation, and perform as a reliable tool for the assessment of inflammation in IUS central reading platforms.

In some embodiments, the AI systems can potentially enhance the dissemination of home patient-based IUS monitoring by assisting the acquisition of high-quality bowel images and the interpretation of the acquired images.

In some embodiments, the ultrasonographic signs of IBD are not limited to bowel wall thickening and include increased blood perfusion (detected by applying Doppler), thickened mesenteric fat, stratification of the bowel wall layers, enlarged mesenteric lymph nodes and attenuated peristalsis.

In some embodiments, IUS can also detect disease penetrative complications (fistula and abscess) and strictures [Ref 22].

In some embodiments, the AI system is used for the detection and recognition of other ultrasonographic signs of IBD, using the convolutional neural network as described herein.

In some embodiments, while a state-of-the-art deep learning network was employed, yet other configurations can be used. It should be understood that those other configurations are included in the scope of the invention. In some embodiments, while manual cropping was used in order to focus the network to the area of interest, an automated cropping software can be used to perform the cropping, for example, by developed new networks that will be able to focus automatically on the ROI. It should be understood that automated cropping is included in the scope of the invention.

In summary, a machine learning framework that is highly accurate in the recognition of bowel wall thickening on intestinal ultrasound images in Crohn's disease was developed. In some embodiments, the neural network extracts meaningful features passing to the SVM which can accurately separate thickened bowel wall from normal bowel.

### Exemplary Automatic Measurements

In some embodiments, the system is configured to perform automatic measurements of features in the images. In some embodiments, the automatic measurements are performed on native non-process images and/or on processed images. In some embodiments, for example, images that when through the segmentation process, are used to measure the colon area in order to proceed to the automatic identification of inflamed colons.

Exemplary Translation into Treatment Methods

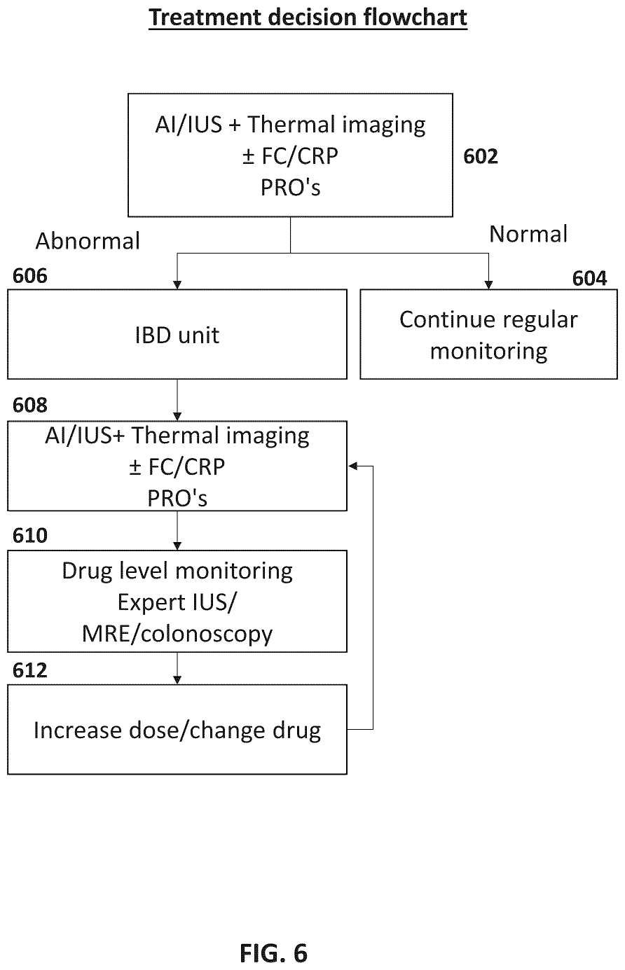

Referring now to FIGS. 6-8, showing flowcharts of exemplary methods performed as translation of the results of the methods disclosed above, into treatment actions.

Referring ow to FIG. 6, showing a flowchart of an exemplary decision of treatment, according to some embodiments of the invention. In some embodiments, AI IUS is performed as disclosed above 602. In some embodiments, additionally thermal imaging is performed. In some embodiments, an assessment of Fecal Calprotectin (FC) and C-reactive protein (CRP) are performed. In some embodiments, Patient Reported Outcome (PRO's) are provided.

In some embodiments, if the results are normal, then the patient is regularly monitored 604, to assess changes in his status.

In some embodiments, if the results are abnormal, the patient is sent to the IBD unit 606, where the examinations are repeated 608 as in 602.

In some embodiments, the patient is provided with the necessary treatment 608, for example, prescribing relevant drugs, directing the patient to an expert in IUS for further examinations, directing the patient to perform MRE and/or colonoscopies, and others.

In some embodiments, the monitoring can incur an increment and/or a change in the dose of the drug 612. Further monitoring and repeated examinations are performed over time (back to 608).

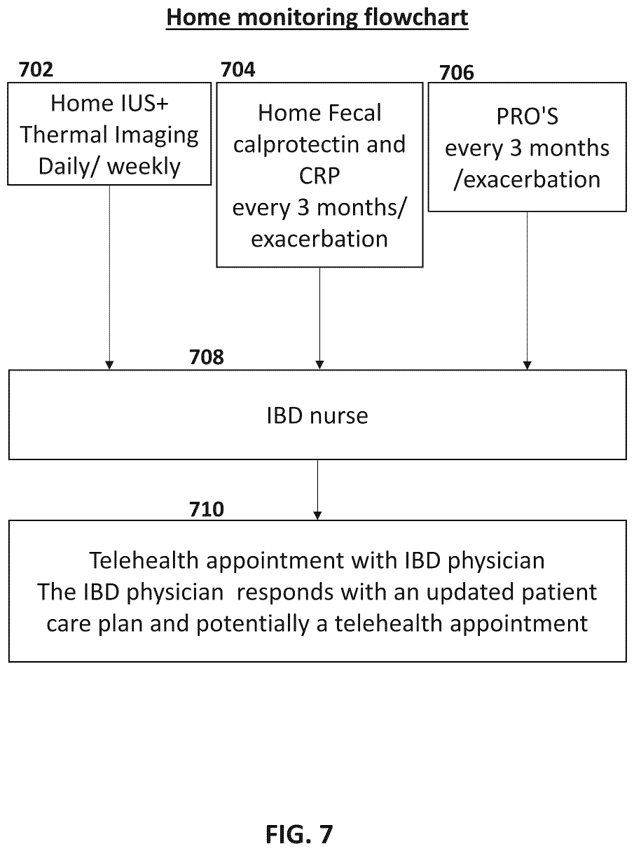

Referring now to FIG. 7, showing a flowchart of an exemplary home monitoring method, according to some embodiments of the invention. In some embodiments, after a patient is diagnosed or when a patient is suspected to have IBD, but the examinations are needed to be performed at home and/or routine examinations are desired to be performed at home, the following actions are performed. In some embodiments, the patient is examined by performing an IUS at home (either by a nurse or by himself), optionally performing a thermal imaging examination 702. In some embodiments, the examination is performed on a daily basis, or a weekly basis, etc. In some embodiments, optionally, a Home Fecal calprotectin and CRP test is performed 704. In some embodiments, the test is performed every 3 months or until exacerbation. In some embodiments, optionally, PRO's are provided 706. In some embodiments, the PRO's are provided every 3 months or until exacerbation. In some embodiments, the results of the tests (702/704/706) are sent and/or collected, for example, by an IBD nurse or any other dedicated personnel 708. In some embodiments, following the analysis of the results, a telehealth appointment with an IBD physician can be scheduled, where the IBD physician can potentially respond with an updated patient care plan and potentially a setting a future telehealth appointment 710.

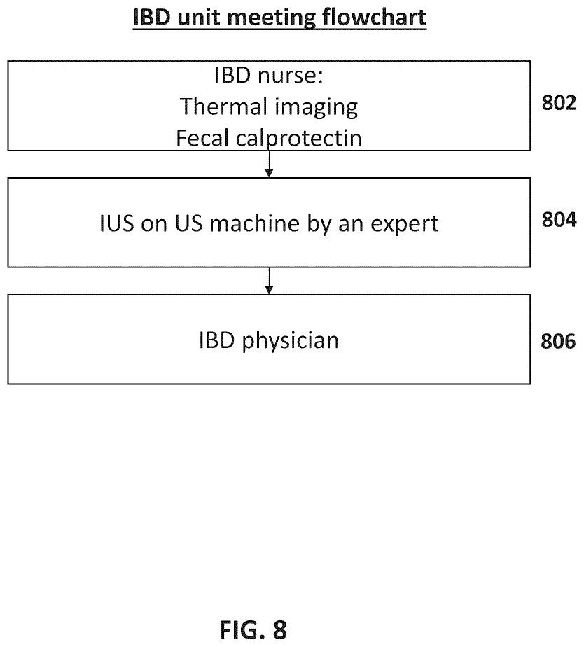

Referring now to FIG. 8, showing a flowchart of an exemplary method of the actions performed in an exemplary IBD unit meeting following the receipt of the results of the examinations, according to some embodiments of the invention. In some embodiments, the IBD nurse presents the thermal images and/or the fecal calprotein results 802. In some embodiments, an IUS on an US machine is performed and/or analyzed by an expert 804. In some embodiments, the results are sent to an IBD physician 806.

As used herein with reference to quantity or value, the term “about” means “within +20% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental and/or calculated support in the following examples.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

1. Wilkens R, Dolinger M, Burisch J, et al. Point-of-Care Testing and Home Testing: Pragmatic Considerations for Widespread Incorporation of Stool Tests, Serum Tests, and Intestinal Ultrasound. Gastroenterology. 2022; 162(5): 1476-1492.
2. Maaser C, Maconi G, Kucharzik T, et al. Ultrasonography in inflammatory bowel disease—So far we are? United European Gastroenterol J. 2022; 10(2): 225-232.
3. Maaser C, Sturm A, Vavricka S R, et al. European Crohn's and Colitis Organisation [ECCO] and the European Society of Gastrointestinal and Abdominal Radiology [ESGAR].
4. ECCO-ESGAR Guideline for Diagnostic Assessment in IBD Part 1: Initial diagnosis, monitoring of known IBD, detection of complications. J Crohns Colitis. 2019 1; 13(2): 144-164.
5. Novak K L, Nylund K, Maaser C, et al. Expert Consensus on Optimal Acquisition and Development of the International Bowel Ultrasound Segmental Activity Score [IBUS-SAS]: A Reliability and Inter-rater Variability Study on Intestinal Ultrasonography in Crohn's Disease. J Crohns Colitis. 2021 6; 15(4): 609-616.
6. lvemark J F K F, Hansen T, Goodsall T M, et al. Defining Transabdominal Intestinal Ultrasound Treatment Response and Remission in Inflammatory Bowel Disease: Systematic Review and Expert Consensus Statement. J Crohns Colitis. 2022 10; 16(4): 554-580.
7. Goodsall T M, Jairath V, Feagan B G, et al. Standardisation of intestinal ultrasound scoring in clinical trials for luminal Crohn's disease. Aliment Pharmacol Ther. 2021; 53(8): 873-886.
8. Vaughan R, Tjandra D, Patwardhan A, et al. Toward transmural healing: Sonographic healing is associated with improved long-term outcomes in patients with Crohn's disease. Aliment Pharmacol Ther. 2022 Mar. 28. doi: 10.1111/apt.16892.
9. Kucharzik T, Wittig B M, Helwig U, et al. Use of Intestinal Ultrasound to Monitor Crohn's Disease Activity. Clin Gastroenterol Hepatol. 2017; 15(4): 535-542.
10. Maaser C, Petersen F, Helwig U, et al. Intestinal ultrasound for monitoring therapeutic response in patients with ulcerative colitis: results from the TRUST&UC study. Gut. 2020; 69(9): 1629-1636.
11. Kucharzik T, Wilkens R, Maconi G, et al. Intestinal ultrasound response and transmural healing after ustekinumab induction in Crohn's disease: Week 16 interim analysis of the STARDUST trial substudy. Crohns Colitis 2020; 14: S046-48.
12. Le Berre C, Sandborn W J, Aridhi S, et al. Application of Artificial Intelligence to Gastroenterology and Hepatology. Gastroenterology. 2020; 158(1): 76-94.
13. Marya N B, Powers P D, Chari S T, et al. Utilisation of artificial intelligence for the development of an EUS-convolutional neural network model trained to enhance the diagnosis of autoimmune pancreatitis. Gut. 2021; 70(7): 1335-1344.
14. Udriştoiu A L, Cazacu I M, Gruionu L G, et al. Real-time computer-aided diagnosis of focal pancreatic masses from endoscopic ultrasound imaging based on a hybrid convolutional and long short-term memory neural network model. PLOS One. 2021 28; 16(6): e0251701.
15. Marya N B, Powers P D, Fujii-Lau L, et al. Application of artificial intelligence using a novel EUS-based convolutional neural network model to identify and distinguish benign and malignant hepatic masses. Gastrointest Endosc. 2021; 93(5): 1121-1130.
16. He K, Zhang X, Ren S, et al. Deep residual learning for image recognition. Proceedings of the IEEE Conference on Computer Vision and Pattern Recognition 2016:770-778.
17. Horsthuis K, Bipat S, Bennink R J, et al. Inflammatory bowel disease diagnosed with U S, M R, scintigraphy, and C T: meta-analysis of prospective studies. Radiology. 2008; 247(1): 64-79.
18. Miles A, Bhatnagar G, Halligan S, et al. Magnetic resonance enterography, small bowel ultrasound and colonoscopy to diagnose and stage Crohn's disease: patient acceptability and perceived burden. Eur Radiol. 2019; 29(3): 1083-1093.
19. Kucharzik T, Petersen F, Maaser C. Bowel Ultrasonography in Inflammatory Bowel Disease. Dig Dis. 2015; 33 Suppl 1:17-25.
20. Calabrese E, Kucharzik T, Maaser C, et al. Real-time Interobserver Agreement in Bowel Ultrasonography for Diagnostic Assessment in Patients With Crohn's Disease: An International Multicenter Study. Inflamm Bowel Dis. 2018 16; 24(9): 2001-2006.
21. De Voogd F, Wilkens R, Gecse K, et al. A Reliability Study: Strong Inter-Observer Agreement of an Expert Panel for Intestinal Ultrasound in Ulcerative Colitis. J Crohns Colitis. 2021 2; 15(8): 1284-1290.
22. Kucharzik T, Tielbeek J, Carter D, et al. ECCO-ESGAR Topical Review on Optimizing Reporting for Cross-Sectional Imaging in Inflammatory Bowel Disease. J Crohns Colitis. 2022 10; 16(4): 523-543.

1. A system, comprising:
a computing device, the computing device comprising:
i. a communication interface configured to receive ultrasound echo data of at least a portion of an intestine comprising a plurality of ultrasound images of said at least a portion of an intestine;
ii. a memory configured for:
A. storing a representation of a inflammation indicating protocol for intestinal inflammation IBD predicting an intestinal inflammation for a patient based on the presence of at least one inflammation indicating sign of a plurality of inflammation indicating signs in ultrasound images captured from a patient;
iii. a circuitry configured to:
B. detecting one or more inflammation indicating signs among the plurality of inflammation indicating signs in ultrasound images of at least a portion of an intestine;
C. based on the identified one or more inflammation indicating signs, evaluate the represented inflammation indicating protocol for intestinal inflammation to obtain a tentative indication of intestinal inflammation of the person.

2. The system according to claim 1, wherein said plurality of inflammation indicating signs comprise one or more of bowel wall thickening greater than 3 mm, increased bowel wall blood flow as detected by doppler, mesenteric fat proliferation, disappearance of bowel wall layers, enlarged lymph nodes in the mesenteric fat and appearance of intraluminal complications and/or extramural complications.

3. The system according to claim 1, comprising an ultrasound sensing device.

4. The system according to claim 1, wherein one of the plurality of inflammation indicating signs is a compound sign that depends on two or more other of the plurality of inflammation indicating signs, wherein the processor is further configured for determining whether the compound sign is present or absent based on whether the inflammation indicating signs on which the compound sign depends are identified as present or absent.

5. (canceled)

6. The system according to claim 1, wherein said process further comprises instructions for performing a segmentation process on said each received ultrasound image.

7. One or more instances of computer-readable media collectively having contents configured to cause a computing system to perform a method, the method comprising:
a. accessing a set of inflammation indicating signs of intestinal inflammation involved in a inflammation indicating protocol for intestinal inflammation;
b. until the presence or absence of each of the inflammation indicating signs of intestinal inflammation of the set has been identified in ultrasound images of a patient:
i. causing an ultrasound image of at least a part of an intestine to be captured from the patient;
ii. detecting one or more inflammation indicating signs among the set of inflammation indicating signs in ultrasound images of at least a portion of an intestine;
iii. evaluating the inflammation indicating protocol for intestinal inflammation with respect to the identified presence or absence of each of the set of inflammation indicating signs of intestinal inflammation to obtain a preliminary indication for intestinal inflammation.

8. The one or more instances of computer-readable media according to claim 7, wherein said set of inflammation indicating signs comprise one or more of bowel wall thickening greater than 3 mm, increased bowel wall blood flow as detected by doppler, mesenteric fat proliferation, disappearance of bowel wall layers, enlarged lymph nodes in the mesenteric fat and appearance of intraluminal complications and/or extramural complications.

9. The one or more instances of computer-readable media according to claim 7, the method further comprising training at least one machine learning model to identify the presence or absence of one or more inflammation indicating signs of the set.

10. The one or more instances of computer-readable media according to claim 7, the method further comprising causing the obtained preliminary indication to be displayed.

11. The one or more instances of computer-readable media according to claim 7, wherein one of the set of inflammation indicating signs is a compound sign that depends on two or more other of the set of inflammation indicating signs, the method further comprising determining whether the compound sign is present or absent based on whether the inflammation indicating signs on which the compound sign depends are identified as present or absent.

12. The one or more instances of computer-readable media according to claim 7, wherein the method further comprises applying a segmentation process to said captured ultrasound image.

13. A method in a computing system, the method comprising:
a. accessing a set of inflammation indicating signs of intestinal inflammation involved in a inflammation indicating protocol for intestinal inflammation;
b. receiving a plurality of ultrasound images of at least a portion of an intestine;
c. detecting one or more inflammation indicating signs among the set of inflammation indicating signs in ultrasound images of at least a portion of an intestine;
iii. evaluating the inflammation indicating protocol with respect to the determined inflammation indicating signs to obtain a preliminary indication.

14. The method according to claim 13, wherein said set of inflammation indicating signs comprise one or more of bowel wall thickening greater than 3 mm, increased bowel wall blood flow as detected by doppler, mesenteric fat proliferation, disappearance of bowel wall layers, enlarged lymph nodes in the mesenteric fat and appearance of intraluminal complications and/or extramural complications.

15. The method according to claim 13, wherein the detecting comprises training at least one convolutional neural network to identify a presence or absence of one or more inflammation indicating signs of the set.

16. The method according to claim 13, further comprising causing the obtained preliminary indication to be displayed.

17. The method according to claim 13, wherein one of the set of inflammation indicating signs is a compound sign that depends on two or more other of the set of inflammation indicating signs, the method further comprising: determining whether the compound sign is present or absent based on whether the inflammation indicating signs on which the compound sign depends are identified as present or absent.

18. The method according to claim 13, wherein at least one of the one or more convolutional neural networks comprises a Mobile U-Net.

19. The method according to claim 13, wherein the method further comprises applying a segmentation process to said captured ultrasound image.

20. The system according to claim 3, wherein the circuitry is configured to applying one or more trained neural networks to each received ultrasound image of said at least a portion of an intestine to identify at least one inflammation indicating sign of the plurality of inflammation indicating signs of intestinal inflammation as present or absent in the received ultrasound image, until the plurality of signs are all identified as present or absent in the person, to detect one or more inflammation indicating signs among the plurality of inflammation indicating signs.

21. The system according to claim 20, wherein the circuitry is configured to cause the capture of additional ultrasound images from the person by the ultrasound sensing device.

22. The system according to claim 1, wherein the circuitry is configured evaluate the represented inflammation indicating protocol for intestinal inflammation to obtain a tentative indication of inflammatory bowel disease (IBD) of the person.

23. The system according to claim 1, comprising a display device configured to causing the tentative indication of intestinal inflammation of the person to be displayed.
